(19) United States
(12) Patent Application Publication
(10) Pub. No.: US 2019/0367647 A1
(43) Pub. Date: Dec. 5, 2019

(54) FUNCTIONALIZED POLY(2,6-DIMETHYL PHENYLENE OXIDE) OLIGOMERS CONTAINING DICYCLOPENTADIENE, METHOD OF PRODUCING THE SAME AND USE THEREOF

(71) Applicant: CPC Corporation, Taiwan, KAOHSIUNG CITY (TW)

(72) Inventors: Sheng-De Li, CHIAYI CITY (TW); Ming-Yu Huang, CHIAYI CITY (TW); Jui-Fu Kao, CHIAYI CITY (TW); Way-Chih Hsu, CHIAYI CITY (TW); Jann-Chen Lin, CHIAYI CITY (TW); Yih-Ping Wang, CHIAYI CITY (TW)

(21) Appl. No.: 16/383,055

(22) Filed: Apr. 12, 2019

(30) Foreign Application Priority Data
Jun. 5, 2018 (TW) 107119357

(51) Int. Cl. C08F 120/30; C08F 136/14; C09D 133/14; C09D 147/00; C09J 133/14; C09J 147/00; C07C 43/295

(52) U.S. Cl. CPC C08F 120/30; C08F 136/14; C09D 133/14; C07C 2603/68; C09J 133/14; C09J 147/00; C07C 43/295; C09D 147/00

(57) ABSTRACT

The invention discloses functionalized poly(2,6-dimethyl phenylene oxide oxide) oligomers containing dicyclopentadiene, a method of producing the same and use thereof. The cured products of the functionalized poly(2,6-dimethyl phenylene oxide oxide) oligomers of the invention exhibit low dielectric constant, low dissipation, and high glass transition temperature. As the functionalized poly(2,6-dimethyl phenylene oxide oxide) oligomers of the invention have number-average molecular weight ranging from 2500 to 6000 g/mol, the substrate made of theses functionalized poly(2,6-dimethyl phenylene oxide oxide) oligomers can pass the pressure cook test. Besides, the low dissipation factor characteristic the functionalized poly(2,6-dimethyl phenylene oxide oxide) oligomers of the invention can only be demonstrated at number-average molecular weight higher than 2500 g/mol.

### FIELD OF THE INVENTION

The present invention relates to functionalized poly(2,6-dimethyl phenylene oxide) oligomers containing dicyclopentadiene (DCPD), method of producing the same and use thereof. Comparing to those of the commercial functionalized poly(2,6-dimethyl phenylene oxide), the cured products of the functionalized poly(2,6-dimethyl phenylene oxide) oligomers provided by the present invention have lower dielectric constant and dielectric loss, which could be used as resin materials for making high frequency substrates.

### BACKGROUND OF THE INVENTION

The technical background with respect to the present disclosure refers to the technical articles as follows.
[1] U.S. Pat. No. 8,791,214;
[2] U.S. Pat. No. 7,329,708;
[3] S. Fisher, H. G., M. Jeevanath, E. Peters, SABIC Innovative Plastics In Polyphenylene Ether Macromonomer: X. Vinyl Terminated Telechelic Macromers, 69th Annual Technical Conference of the Society of Plastics Engineers 2011 (ANTEC 2011), Boston, Mass., USA, 1-5 May, 2011; pp 2819-2822;
[4] E. N. Peters, A. K., E. Delsman, H. Guo, A. Carrillo, G. Rocha In Society of Plastics Engineers Annual Technical Conference (ANTEC 2007): Plastics Encounter, Cincinnati, Ohio, 6-11 May, 2007; Curran Associates, Inc.; pp 2125-2128;
[5] E. N. Peters, S. M. F., H. Guo, C. Degonzague, R. Howe. In 68th Annual Technical Conference of the Society of Plastics Engineers 2010 (ANTEC 2010), Orlando, Fla., USA., 16-20 May, 2010; Curran Associates, Inc. (August 2010);
[6] Edward N. Peters, S. M. F., Hua Guo In Polyphenylene Ether Macromonomers. XI. Use in Non-Epoxy Printed Wiring Boards, IPC APEX EXPO 2012, San Diego, Calif., USA., 28 February-1 March, 2012; Curran Associates, Inc.;
[7] Leu, T. S.; Wang, C. S., J. Appl. Polym. Sci. 2004, 92, 410;
[8] Hwang, H.-J.; Li, C.-H.; Wang, C.-S. Polymer International 2006, 55, (11), 1341-1349;
[9] Hwang, H.-J.; Lin, C.-Y.; Wang, C.-S. Journal of Applied Polymer Science 2008, 110, (4), 2413-2423;
[10] Hwang, H.-J.; Li, C.-H.; Wang, C.-S. Journal of Applied Polymer Science 2005, 96, (6), 2079-2089;
[11] Patent/Publication Number 201723130 of the Intellectual Property Office, MOEA, R.O.C.

With the advance of semiconductor technology and downsizing of electronic components, PCB trace width and the trace spacing are getting shorter and shorter, which leads to more crosstalk among traces and propagation delay in traces and dielectric layers, so the electrical properties of dielectric layers play an important role in PCB performance. A dielectric layer with lower dielectric constant (Dk) and lower dielectric loss (Df) contributes to reduce signal loss and increase transmission rate in PCB. Thus, there have been many patents relating to development of low dielectric resin materials to conform to the current demand.

Epoxy resin, which has many advantages such as cheap, both insulation and thermal properties of its cured product are good, is the most used material for dielectric layers. However, the rapid development of resin materials in recent years revealed that the dielectric properties of epoxy resin were not easy to be improved because the highly polar secondary alcohol would be generated after ring-opening polymerization (ROP) of epoxy resin. In 2014, Kan Takeuchi et al. [1] disclosed that esterifying phenolic compounds such as phenol novolac (PN), dicyclopentadiene phenol novolac (DCPDPN) with a monofunctional or bifunctional acyl chloride to give an active ester resin, and then curing the active ester resin with an epoxy resin, HP7200; the epoxy resin would react with the active ester through transesterification during the ring-opening process and the highly polar secondary alcohols weren't generated after curing, which was beneficial to decrease the dielectric constant (Dk). However, after the epoxy resin reacted with the active ester, the hydroxyl group of the ring-opened epoxy resin was replaced by the formed ester group, so there was less intermolecular hydrogen bond which led to a decrease in glass transition temperature (Tg) of the cured product.

Poly(2,6-dimethyl-1,4-phenylene oxide) (PPO), one of the big five engineering plastics, has many advantages such as high glass transition temperature, good resistance to acids and alkalis, and high impact resistance, etc. Besides, PPO exhibits excellent electrical properties and has gradually attracted much attention in recent years because of its low polar and high hydrophobic structure. However, the conventional PPO resin has high molecular weight which makes it have poor solubility and over high viscosity. Also, using PPO resin with high molecular weight as a hardener for epoxy resin easily lead to a phase separation problem of the cured product, and the applications are limited. Thus, there have been many patents regarding development of PPO resin with low molecular weight for improving the processability. In 2008, Birsak et al., from General Electric Company (GE), USA, developed a series of PPO oligomers containing different core functional groups by oxidative coupling polymerization, and modified the terminal phenolic groups to obtain a series of PPO oligomers, as shown in chemical equation (1) [2]. In 2011, Peters et al. [3-6] modified the terminal phenolic groups of PPE-M, a commercial product of SABIC which is also known as Noryl® SA90, to give the terminal of PPE-M contain unsaturated double bonds, as shown in chemical equation (2). As PPE-M is incorporated with terminal methacrylate groups (like M-PPE-M, as shown in chemical equation (2)), the product name of it is NORYL™ Resin SA 9000. If the terminal groups of PPE-M are like VB-PPE-M, as shown in chemical equation (2), the product name of it is OPE-2st. According to the result of the present disclosure, the glass transition temperature (Tg) of the cured product prepared by SA9000 and epoxy resin is 226° C. which is pretty close to the solder temperature commonly used today. It may cause the substrate to bend after being heated, which is not conducive to making double-sided PCB. Besides, the specimen is broken after being heated above the glass transition temperature to reveal that the mechanical properties and dimensional stability of the material are poor at high temperature. Thus, incorporating a structure which can enhance the thermomechanical properties but not reduce the dielectric properties into PPO is what the market needs. (Polar groups increase the glass transition temperature by intermolecular force, but also deteriorate the dielectric properties because of the high polarity of them.)

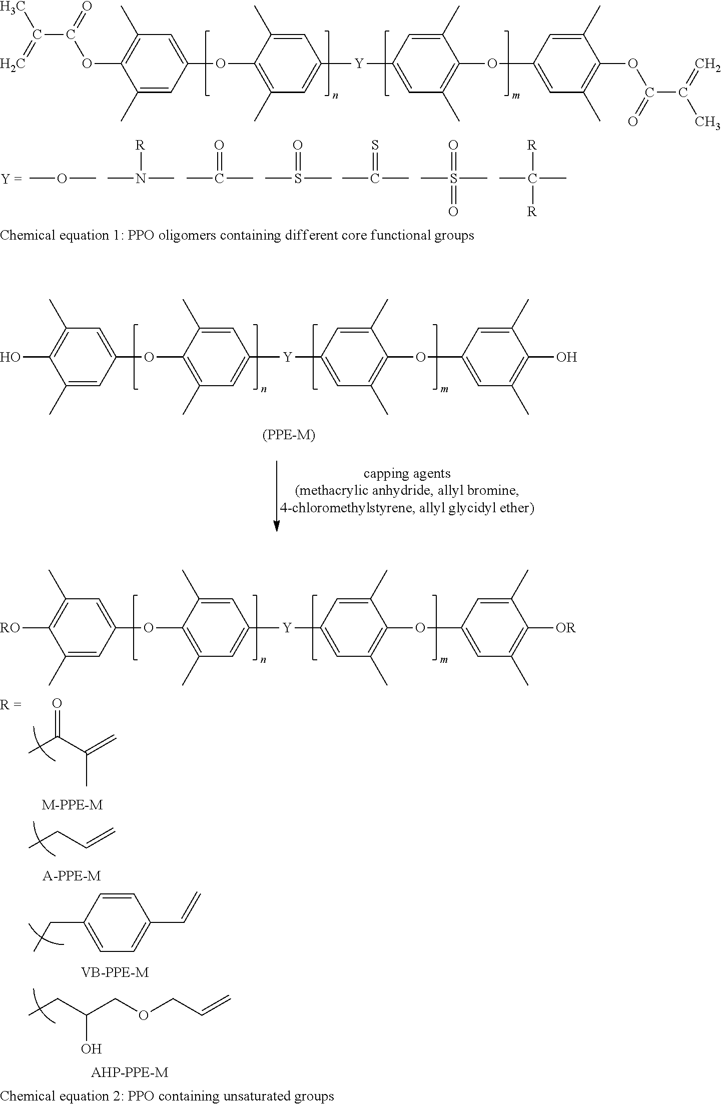

According to the above literatures, the current developments of PPO mostly tend to enhance the performance of PPO copolymers via various modifications of the terminal groups. However, only modifications of the terminal groups result in a limited improvement on PPO performance. DCPD, a by-product of petroleum cracking derived from C5 fraction, is easily separated due to its high boiling point. DCPD contains both of the rigid bicyclic and aliphatic structure; hence, DCPD derivatives exhibit excellent thermal properties and dielectric properties. From 2006 to 2008, Hwang et al. developed a series of the DCPD derivatives including bismaleimide, benzoxazine, and cyanate ester etc. [8-10], and the cured products of them exhibited excellent glass transition temperatures and exceptional dielectric properties. Thus, the present disclosure combines modification of the terminal groups of PPO with incorporation DCPD into PPO structure to give functionalized PPO oligomers which can not only self-cure but also be used as epoxy resin hardeners, and the cured products of them have excellent thermal and electrical properties.

The Taiwan Patent, TW201723130, [11] disclosed a polyphenylene ether oligomer which is similar to the oligomers provided by the present invention. However, TW201723130 emphasized that the solubility of the polyphenylene ether oligomer in acetone was poor when the number-average molecular weight (Mn) of the polyphenylene ether oligomer was higher than 2000 g/mol, so it claimed that the efficient number-average molecular weight of the polyphenylene ether oligomer limited from 400 to 2000 g/mol to have better processability in acetone. However, acetone is not a commonly used solvent in the industry. Also, TW201723130 didn't disclose the glass transition temperature of the polyphenylene ether oligomer as to prove that the polyphenylene ether oligomer has high thermal resistance as it said. Thus, the polyphenylene ether oligomer provided by TW201723130 may not such useful. However, the substrates made from the functionalized poly(2,6-dimethyl phenylene oxide) oligomers in the present invention has been certified and proven to can pass the pressure cook test (PCT) plus 288° C. solder dipping test; only when the oligomers had the number-average molecular weight higher than 2500 g/mol. Besides, according to the data of the electrical properties, the oligomers with too low molecular weight didn't exhibit low dielectric loss characteristic like polyphenylene ether; only the oligomers with the number-average molecular weight higher than 2500 g/mol exhibited the characteristics of low dielectric constant and low dielectric loss.

### SUMMARY OF THE INVENTION

It is therefore an object of the present invention to provide functionalized poly(2,6-dimethyl phenylene oxide) oligomers containing dicyclopentadiene (DCPD), method of producing the same and use thereof. The cured products of the functionalized poly(2,6-dimethyl phenylene oxide) oligomers in the present invention have lower dielectric constant and dielectric loss compared to those of the commercial poly(2,6-dimethyl phenylene oxide) oligomers; thus, the oligomers provided by the present invention can be used as resin materials for high frequency substrate as well as be used in other high temperature resistance applications.

The present invention uses bisphenol monomer, prepared from DCPD, as a starting material to obtain poly(2,6-dimethyl phenylene oxide) oligomers with low molecular weight via oxidative coupling polymerization by using a suitable solvent, and then incorporating unsaturated double bond into the terminals of the oligomers. The cured products with low dielectric properties can be obtained after heating the oligomers.

The advantages and spirit with respect to the functionalized poly(2,6-dimethyl phenylene oxide) oligomers containing dicyclopentadiene are further explained in embodiments as follows.

### BRIEF DESCRIPTION OF THE DRAWINGS

The techniques of present invention would be more understandable from the detailed description given herein below and the accompanying figures are provided for better illustration, and thus description and figures are not limitative for present invention, and wherein:

FIG. 1 is a ¹H-NMR spectrum of the oligomer III-mma in Embodiment 11.

FIG. 2 is a MALDI TOF mass spectrum of the oligomer III-mma in Embodiment 11.

FIG. 3 is a ¹H-NMR spectrum of the oligomer IV-mma in Embodiment 15.

FIG. 4 is a MALDI TOF mass spectrum of the oligomer IV-mma in Embodiment 15.

FIG. 5 is a ¹H-NMR spectrum of the oligomer III-vbe in Embodiment 19.

FIG. 6 is a ¹H-NMR spectrum of the oligomer IV-vbe in Embodiment 23.

FIG. 7 is a dynamic mechanical analysis diagram of the cured products of the functionalized poly(2,6-dimethyl phenylene oxide) oligomers in the present invention.

### DETAILED DESCRIPTION OF THE PREFERRED EMBODIMENT

In the following detailed description, for purposes of explanation, numerous specific details are set forth in order to provide a thorough understanding of the disclosed embodiments. It will be apparent, however, that one or more embodiments may be practiced without these specific details.

First, dicyclopentadiene (DCPD) is reacted with a phenol compound such as 2,6-dimethylphenol (2,6-DMP) or 2,3,6-trimethylphenol (2,3,6-TMP) and catalyzed by a Lewis acid catalyst at a controlled temperature to obtain the bisphenol monomer I or II, respectively. The Lewis acid can be BF₃ or aluminium halides, and the aluminum halides can be aluminium trichloride, aluminium tribromide, ethyl aluminium dichloride, and diethylaluminium chloride. The controlled temperature ranges from 80 to 150° C., and the mole ratio of DCPD to phenol is 1:2-1:10. The reaction was named for the core reaction.

The bisphenol monomer I (or II) is reacted with 2,6-DMP through oxidative coupling polymerization at a controlled temperature and an oxygen atmosphere with a suitable solvent in the presence of copper catalyst and amine catalyst to obtain the poly(2,6-dimethyl phenylene oxide) bisphenol oligomer III (or IV), as shown in chemical equation 3 where m and n each independently represents a natural number. The pressure of the oxygen atmosphere is from 14 psi to 150 psi, and the proportion of the oxygen content under the oxygen atmosphere is from 1% to 100%. The suitable solvent is methanol/water co-solvent, and wherein the water content is from 0% to 30%. The controlled temperature is in the range of 0-70° C. and the reaction time is from 1 hour to 4 hours. The copper catalyst can be CuCl, CuCl₂, CuBr, CuBr₂ and mixtures thereof. The amine catalyst is tertiary amine ((C₂H₅)₃N) or dialkylaminopyridine. The alkyl of the dialkylaminopyridine is C₁-C₆ alkyl group. The feed mole ratio of bisphenol monomer I (or II) to 2,6-DMP is 1:2˜1:10.

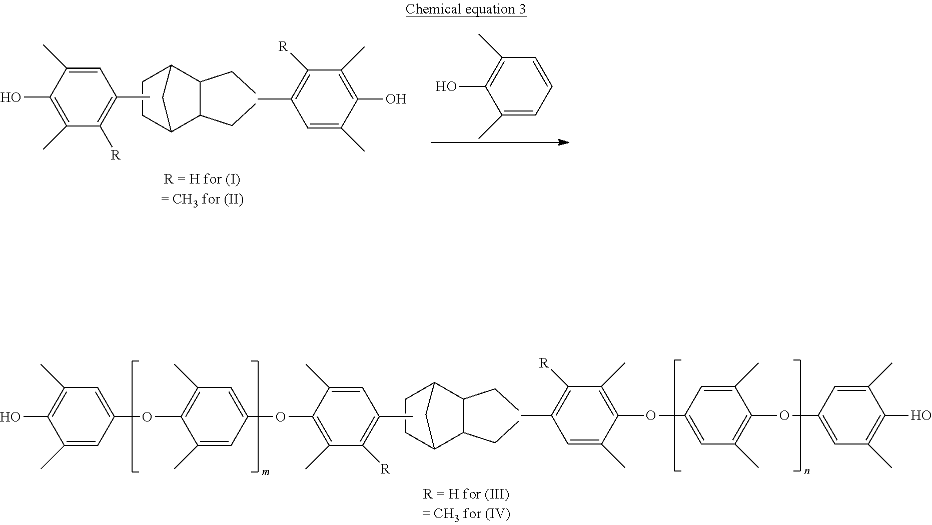

Next, the bisphenol oligomer III (or IV) is reacted with methacrylic anhydride or vinylbenzyl halide through the modifications of the terminal hydroxyl groups at a controlled temperature in the presence of an alkaline catalyst to obtain the functionalized poly(2,6-dimethyl phenylene oxide) oligomer III-mma (or IV-mma) and III-vbe (or IV-vbe) containing unsaturated groups, respectively and the reaction is shown as chemical equation 4. The vinylbenzyl halide is selected from o-vinylbenzyl chloride, m-vinylbenzyl chloride, p-vinylbenzyl chloride, o-vinylbenzyl bromide, m-vinylbenzyl bromide, p-vinylbenzyl bromide and mixtures thereof. The alkaline catalyst is selected from potassium carbonate (K₂CO₃), sodium carbonate (Na₂CO₃), potassium hydroxide (KOH), sodium hydroxide (NaOH), sodium bicarbonate (NaHCO₃), sodium acetate, 4-dimethylamino pyridine, pyridine and mixtures thereof. The controlled temperature is in the range of 45-100° C.

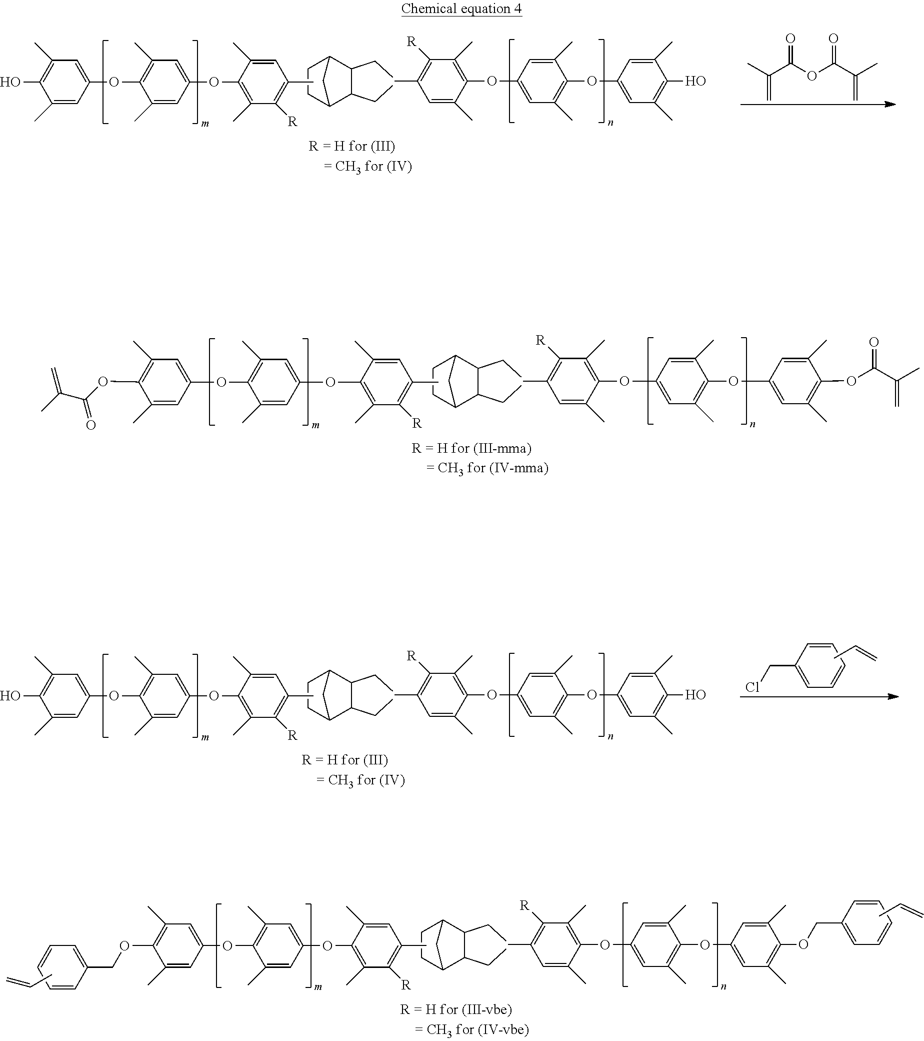

Lastly, a curing reaction of the functionalized poly(2,6-dimethyl phenylene oxide) oligomer III-mma, IV-mma, III-vbe or IV-vbe containing unsaturated groups is carried out by using peroxides as an initiator to obtain the cured products having low dielectric constant, low dielectric loss, and high glass transition temperature. Alternatively, the poly(2,6-dimethyl phenylene oxide) oligomer III-mma or IV-mma is copolymerized with epoxy resin to obtain a copolymer, respectively.

### Embodiment 1: Synthesis of PPO Bisphenol Oligomer III Under Atmospheric Pressure

Bisphenol monomer I and PPO bisphenol oligomer III were prepared as described below:

141.65 g (151.2×7.143 millimole) of 2,6-DMP and 3.25 g of AlCl₃, as a Lewis acid catalyst, were added into a 500 mL three-necked flask. The mixture was stirred and heated to 120° C. under nitrogen atmosphere. Next, 20 g (151.2 millimole) of DCPC was slowly added into the 500 mL three-necked flask and the reaction time was for 2 hours. After the reaction was complete, 150 g of water was added into the reaction solution to stop the reaction, and then diluted with toluene. The diluted solution was subsequently washed with water several times until neutral pH. The organic phase was collected and then filtered to remove the salt and catalyst. The 2,6-DMP and toluene in the organic phase was removed by vacuum distillation at 200° C., and then the bisphenol monomer I was obtained.

Subsequently, 0.18 g (1.818 millimole) of CuCl, 1.2 g (1.818×5.5 millimole) of dimethylamino pyridine (DMAP), 18.6 mL of MeOH, and 1.5 mL of H₂O were added into a 250 mL three necked flask. The mixed solution was continuously stirred for 15 minutes to form a catalyst solution under oxygen atmosphere. Additionally, 2.31 g (6.141 millimole) of the bisphenol monomer I and 3.00 g (6.141×4 millimole) of 2,6-DMP were pre-dissolved in 30 mL of MeOH and then added into the catalyst solution to carry out the reaction for 4 hours under oxygen atmosphere. After the reaction was complete, a filter cake was obtained by filtration, and was subsequently neutralized, washed, purified, and dried to give a light tan powder in around 61% yield.

According to the ¹H-NMR spectrum of the PPO bisphenol oligomer III, the peak at 6.9 ppm corresponded to the benzene ring at the core of DCPD and the peak at 4.2 ppm corresponded to both sides of the terminal phenolic group were observed. The number-average molecular weight of the PPO bisphenol oligomer III was 3845 g/mol and the weight-average molecular weight of the PPO bisphenol oligomer III was 5149 g/mol, which were analyzed by gel permeation chromatography (GPC).

### Embodiment 2: High-Pressure Process (1) for Synthesis of PPO Bisphenol Oligomer III

PPO bisphenol oligomer III can be prepared by using high-pressure reactor as described in detail below:

2.86 g (20 millimole) of CuBr, 12 g (18.18×5.5 millimole) of DMAP, 186 mL of MeOH and 15 mL of H₂O were mixed and added into a 600 mL high-pressure reactor. Then, 23.1 g (61.41 millimole) of the bisphenol monomer I, prepared from embodiment 1, 30.0 g (6.141×4 millimole) of 2,6-DMP were pre-dissolved in 300 mL of MeOH and added into the 600 mL high-pressure reactor. After locked, the 600 mL high-pressure reactor was placed in a thermostatic bath to keep the temperature at 15° C. and air was introduced into the 600 mL high-pressure reactor at a high pressure of 98 psi (exhaust: 15 g/h). The mixture was continuously stirred and the reaction time was for an hour. After the reaction was complete, a filter cake was obtained by filtration, and was subsequently neutralized, washed, purified, and dried to give a light tan powder in 81% yield which was much higher than that of embodiment 1(61%). The number-average molecular weight was 4058 g/mol and the weight-average molecular weight was 5231 g/mol, which were analyzed by gel permeation chromatography (GPC).

### Embodiment 3: High-Pressure Process (2) for Synthesis of PPO Bisphenol Oligomer III

PPO bisphenol oligomer III can be prepared by bisphenol monomer I with an excess and unreacted 2,6-DMP after the core reaction as described in detail below:

110.79 g (151.2×6 millimole) of 2,6-DMP and 3.25 g of AlCl₃, as a Lewis acid catalyst, were added into a 500 mL three-necked flask. The mixture was stirred and heated to 120° C. under nitrogen atmosphere. Next, 19.96 g (151.2 millimole) of DCPD was slowly added into the 500 mL three-necked flask and the reaction was for 2 hours. After the reaction was complete, 100 g of water was added into the reaction solution to stop the reaction, and then diluted with toluene. The diluted solution was subsequently washed with water several times until neutral pH. The organic phase was collected, and then filtered to remove the salt and catalyst. The toluene in the organic phase was removed by vacuum distillation at 140° C. to obtain a mixture of the unreacted 2,6-DMP and the bisphenol monomer I.

Subsequently, 2.0 g (20 millimole) of CuCl, 5.56 g (55 millimole) of triethylamine, 90 mL of MeOH, and 8.3 mL of H₂O were added into a 600 mL high-pressure reactor and the solution was stirred. Then, 29.5 g of the mixture of the unreacted 2,6-DMP and the bisphenol monomer I was pre-dissolved in 124 mL of MeOH and added into the 600 mL high-pressure reactor. After locked, the 600 mL high-pressure reactor was placed in a thermostatic bath to keep the temperature at 15° C. and air was introduced into the 600 mL high-pressure reactor at a high pressure of 98 psi (exhaust: 15 g/h). The mixture was continuously stirred and the reaction was for an hour. After the reaction was complete, a filter cake was obtained by filtration, and was subsequently neutralized, washed, purified, and dried to give a light tan powder in 86% yield. The number-average molecular weight was 3943 g/mol and the weight-average molecular weight was 5192 g/mol, which were analyzed by gel permeation chromatography (GPC).

### Embodiment 4: Synthesis of PPO Bisphenol Oligomer III

PPO bisphenol oligomer III was prepared as embodiment 1 except that the volume ratio of methanol (mL) to water (mL) was 48.6:5. The number-average molecular weight was 2810 g/mol and the weight-average molecular weight was 3632 g/mol, which were analyzed by gel permeation chromatography (GPC).

### Embodiment 5: Synthesis of PPO Bisphenol Oligomer III

PPO bisphenol oligomer III was prepared as embodiment 1 except that the volume ratio of methanol (mL) to water (mL) was 48.6:10. The number-average molecular weight was 2512 g/mol and the weight-average molecular weight was 3066 g/mol, which were analyzed by gel permeation chromatography (GPC).

### Embodiment 6: Synthesis of PPO Bisphenol Oligomer III

PPO bisphenol oligomer III was prepared as embodiment 1 except that the volume ratio of methanol (mL) to water (mL) was 48.6:0. The number-average molecular weight was 4444 g/mol and the weight-average molecular weight was 9332 g/mol, which were analyzed by gel permeation chromatography (GPC).

Comparison 1: Synthesis of PPO Bisphenol Oligomer III

PPO bisphenol oligomer III was prepared as embodiment 1 except that the volume ratio of methanol (mL) to water (mL) was 48.6:30. The number-average molecular weight was 1719 g/mol and the weight-average molecular weight was 2063 g/mol, which were analyzed by gel permeation chromatography (GPC).

### Embodiment 7: Synthesis of PPO Bisphenol Oligomer IV

Bisphenol monomer II and PPO bisphenol oligomer IV were prepared as described below:

147.10 g (151.2×7.143 millimole) of 2,3,6-TMP and 3.6 mL of BF₃ (in ether), as a Lewis acid catalyst, were added into a 500 mL three-necked flask. The mixture was stirred and heated to 120° C. under nitrogen atmosphere. Next, 20 g (151.2 millimole) of DCPC was slowly added into the 500 mL three-necked flask and the reaction was for 2 hours. After the reaction was complete, 150 g of water was added into the reaction solution to stop the reaction, and then diluted with toluene. The diluted solution was subsequently washed with water several times until neutral pH. The organic phase was collected, and then filtered to remove the salt and catalyst. The 2,6-DMP and toluene in the organic phase was removed by vacuum distillation at 200° C., and then the bisphenol monomer II was obtained.

Subsequently, 0.18 g (1.818 millimole) of CuCl, 1.2 g (1.818×5.5 millimole) of DMAP, 18.6 mL of MeOH, and 1.5 mL of H₂O were added into a 250 mL three necked flask. The mixed solution was continuously stirred for 15 minutes to form a catalyst solution under oxygen atmosphere. Additionally, 2.48 g (6.141 millimole) of the bisphenol monomer II and 3.00 g (6.141×4 millimole) of 2,6-DMP were pre-dissolved in 30 mL of MeOH, and then added into the catalyst solution to carry out the reaction for 4 hours under oxygen atmosphere. After the reaction was complete, a filter cake was obtained by filtration, and was subsequently neutralized, washed, purified, and dried to give a light tan powder in 50.3% yield.

According to the ¹H-NMR spectrum of the PPO bisphenol oligomer IV, the peak at 6.9 ppm corresponded to the benzene ring at the core of DCPD, and the peak at 4.2 ppm corresponded to both sides of the terminal phenolic group were observed. The number-average molecular weight of the PPO bisphenol oligomer IV was 3113 g/mol and the weight-average molecular weight of the PPO bisphenol oligomer IV was 3649 g/mol, which were analyzed by gel permeation chromatography (GPC).

### Embodiment 8: Synthesis of PPO Bisphenol Oligomer IV

PPO bisphenol oligomer IV was prepared as embodiment 7 except that the volume ratio of methanol (mL) to water (mL) was 48.6:5. The number-average molecular weight was 2670 g/mol and the weight-average molecular weight was 3211 g/mol, which were analyzed by gel permeation chromatography (GPC).

### Embodiment 9: Synthesis of PPO Bisphenol Oligomer IV

PPO bisphenol oligomer IV was prepared as embodiment 7 except that the volume ratio of methanol (mL) to water (mL) was 48.6:10. The number-average molecular weight was 2347 g/mol and the weight-average molecular weight was 2581 g/mol, which were analyzed by gel permeation chromatography (GPC).

### Embodiment 10: Synthesis of PPO Bisphenol Oligomer IV

PPO bisphenol oligomer IV was prepared as embodiment 7 except that the volume ratio of methanol (mL) to water (mL) was 48.6:0. The number-average molecular weight was 5312 g/mol and the weight-average molecular weight was 13280 g/mol, which were analyzed by gel permeation chromatography (GPC).

Comparison 2: Synthesis of PPO Bisphenol Oligomer IV

PPO bisphenol oligomer IV was prepared as embodiment 7 except that the volume ratio of methanol (mL) to water (mL) was 48.6:30. The number-average molecular weight was 1583 g/mol and the weight-average molecular weight was 1974 g/mol, which were analyzed by gel permeation chromatography (GPC).

### Embodiment 11: Synthesis of Oligomer III-mma

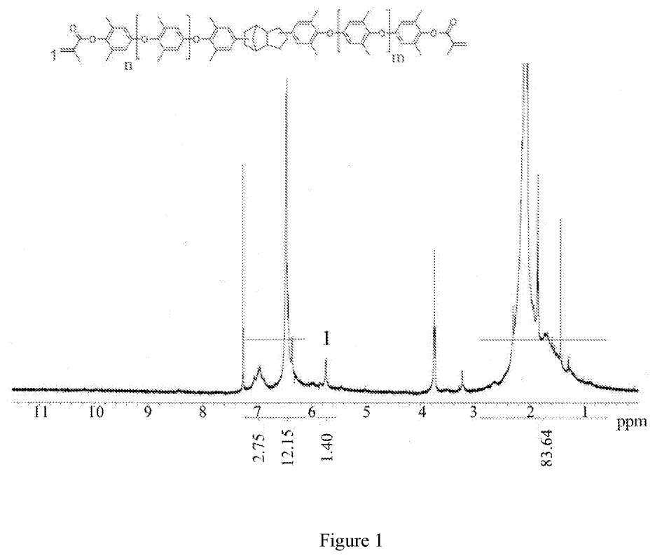

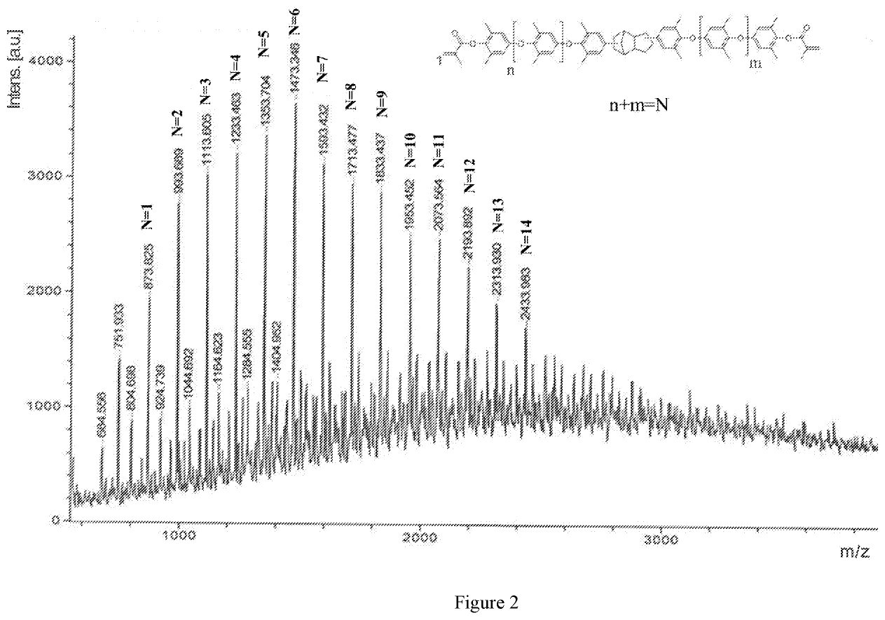

1.00 g of PPO bisphenol oligomer III prepared by embodiment 1, 0.4998 g of methacrylic anhydride, 0.01 g of sodium acetate, and 10 mL of Dimethylacetamide (DMAc) were added into a 150 mL three-necked flask. The mixture was stirred and heated to 75° C. under nitrogen atmosphere. After the reaction lasted for 2 hours, the reaction solution was slowly instilled into 250 mL of saturated salt solution to precipitate and the precipitate solution was filtered to collect the powder. The filter cake was then washed, purified, and dried to give a light tan powder. According to the ¹H-NMR spectrum in FIG. 1, the characteristic peak at 4.2 ppm corresponding to both sides of the terminal phenolic groups of PPO bisphenol oligomer III was disappeared and the characteristic peak at 5.8 ppm corresponding to the unsaturated C═C double bonds of the oligomer III-mma was observed. The number-average molecular weight was 4045 g/mol and the weight-average molecular weight was 5610 g/mol, which were analyzed by gel permeation chromatography (GPC).According to the MALDI TOF mass spectrum in FIG. 2, the molecular weight of the oligomer III-mma was 374+189*2+120*n (refer to the chemical formula in FIG. 2). The peak corresponding to the oligomer III-mma structure with n=1, 2, 3, 4, . . . , 13, 14 can be seen clearly in FIG. 2. The solubility in organic solvents (50 wt %) and the molecular weights of the oligomer III-mma provided by the present invention are summarized in Table 1 and Table 6.

TABLE 1

| | Embodi- | Embodi- | Embodi- | Embodi- | Com- |
|---|---|---|---|---|---|
| | ment | ment | ment | ment | parison |
| | 11 | 12 | 13 | 14 | 3 |
| Methanol | 48.6/1.5 | 48.6/5 | 48.6/10 | 48.6/0 | 48.6/30 |
| (mL)/water | | | | | |
| (mL) | | | | | |
| Mn/PDI | 4045/1.39 | 3130/1.31 | 2711/1.25 | 4563/2.60 | 1920/1.20 |
| Toluene | ++ | ++ | ++ | +− | ++ |
| Butanone | ++ | ++ | ++ | +− | ++ |
| Xylene | ++ | ++ | ++ | +− | ++ |

^{a}++ clear; +− slightly blurred

### Embodiment 12: Synthesis of Oligomer III-mma

The oligomer III-mma was prepared as embodiment 11 except that the PPO bisphenol oligomer III prepared by embodiment 1 was replaced with the PPO bisphenol oligomer III prepared by embodiment 4. The number-average molecular weight was 3130 g/mol and the weight-average molecular weight was 4109 g/mol, which were analyzed by gel permeation chromatography (GPC).

### Embodiment 13: Synthesis of Oligomer III-mma

The oligomer III-mma was prepared as embodiment 11 except that the PPO bisphenol oligomer III prepared by embodiment 1 was replaced with the PPO bisphenol oligomer III prepared by embodiment 5. The number-average molecular weight was 2711 g/mol and the weight-average molecular weight was 3382 g/mol, which were analyzed by gel permeation chromatography (GPC).

### Embodiment 14: Synthesis of Oligomer III-mma

The oligomer III-mma was prepared as embodiment 11 except that the PPO bisphenol oligomer III prepared by embodiment 1 was replaced with the PPO bisphenol oligomer III prepared by embodiment 6. The number-average molecular weight was 4563 g/mol and the weight-average molecular weight was 11864 g/mol, which were analyzed by gel permeation chromatography (GPC).

Comparison 3: Synthesis of Oligomer III-mma

The oligomer III-mma was prepared as embodiment 11 except that the PPO bisphenol oligomer III prepared by embodiment 1 was replaced with the PPO bisphenol oligomer III prepared by comparison 1. The number-average molecular weight was 1920 g/mol and the weight-average molecular weight was 2312 g/mol, which were analyzed by gel permeation chromatography (GPC).

### Embodiment 15: Synthesis of Oligomer IV-mma

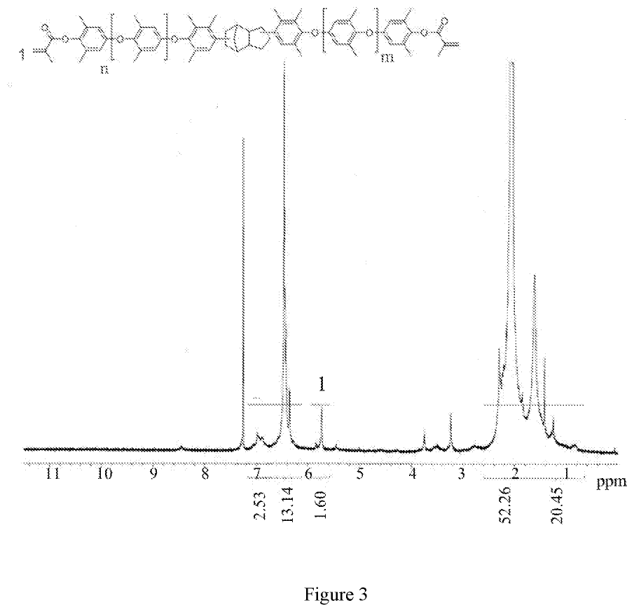

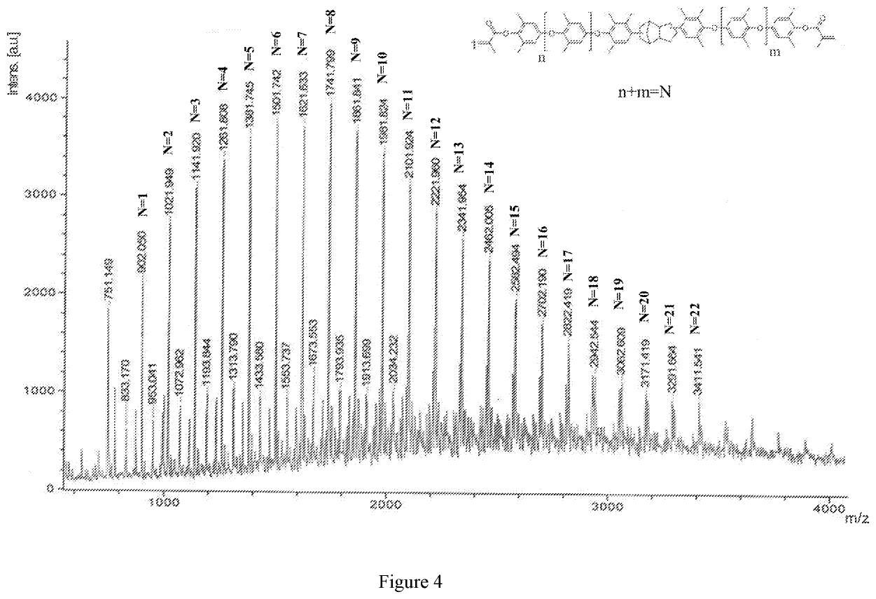

1.00 g of PPO bisphenol oligomer IV prepared by embodiment 7, 0.4998 g of methacrylic anhydride, 0.01 g of sodium acetate, and 10 mL of DMAc were added into a 150 mL three-necked flask. The mixture was stirred and heated to 75° C. under nitrogen atmosphere. After the reaction lasted for 2 hours, the reaction solution was slowly instilled into 250 mL of saturated salt solution to precipitate and the precipitate solution was filtered to collect the powder. The filter cake was then washed, purified, and dried to give a light tan powder. According to the ¹H-NMR spectrum in FIG. 3, the characteristic peak at 4.2 ppm corresponding to both sides of the terminal phenolic groups of PPO bisphenol oligomer IV was disappeared and the characteristic peak at 5.8 ppm corresponding to the unsaturated C═C double bonds of the oligomer IV-mma was observed. The number-average molecular weight was 3833 g/mol and the weight-average molecular weight was 5023 g/mol, which were analyzed by gel permeation chromatography (GPC). The peak corresponding to the oligomer IV-mma structure with n=1, 2, 3, 4, . . . , 13, 14 can be seen clearly from the MALDI TOF mass spectrum in FIG. 4. The solubility in organic solvents (50 wt %) and the molecular weights of the oligomer IV-mma provided by the present invention are summarized in Table 2 and Table 6.

TABLE 2

| | Embodi- | Embodi- | Embodi- | Embodi- | Com- |
|---|---|---|---|---|---|
| | ment | ment | ment | ment | parison |
| | 15 | 16 | 17 | 18 | 4 |
| Methanol | 48.6/1.5 | 48.6/5 | 48.6/10 | 48.6/0 | 48.6/30 |
| (mL)/water | | | | | |
| (mL) | | | | | |
| Mn/PDI | 3833/1.31 | 2950/1.35 | 2656/1.14 | 5451/2.70 | 1712/1.26 |
| Toluene | ++ | ++ | ++ | +− | ++ |
| Butanone | ++ | ++ | ++ | +− | ++ |
| Xylene | ++ | ++ | ++ | +− | ++ |

^{a}++ clear; +− slightly blurred

### Embodiment 16: Synthesis of Oligomer IV-mma

The oligomer IV-mma was prepared as embodiment 15 except that the PPO bisphenol oligomer IV prepared by embodiment 7 was replaced with the PPO bisphenol oligomer IV prepared by embodiment 8. The number-average molecular weight was 2951 g/mol and the weight-average molecular weight was 3991 g/mol, which were analyzed by gel permeation chromatography (GPC).

### Embodiment 17: Synthesis of Oligomer IV-mma

The oligomer IV-mma was prepared as embodiment 15 except that the PPO bisphenol oligomer IV prepared by embodiment 7 was replaced with the PPO bisphenol oligomer IV prepared by embodiment 9. The number-average molecular weight was 2656 g/mol and the weight-average molecular weight was 3021 g/mol, which were analyzed by gel permeation chromatography (GPC).

### Embodiment 18: Synthesis of Oligomer IV-mma

The oligomer IV-mma was prepared as embodiment 15 except that the PPO bisphenol oligomer IV prepared by embodiment 7 was replaced with the PPO bisphenol oligomer IV prepared by embodiment 10. The number-average molecular weight was 5451 g/mol and the weight-average molecular weight was 14717 g/mol, which were analyzed by gel permeation chromatography (GPC).

Comparison 4: Synthesis of Oligomer IV-mma

The oligomer IV-mma was prepared as embodiment 15 except that the PPO bisphenol oligomer IV prepared by embodiment 7 was replaced with the PPO bisphenol oligomer IV prepared by comparison 2. The number-average molecular weight was 1712 g/mol and the weight-average molecular weight was 2154 g/mol, which were analyzed by gel permeation chromatography (GPC).

### Embodiment 19: Synthesis of Oligomer III-vbe

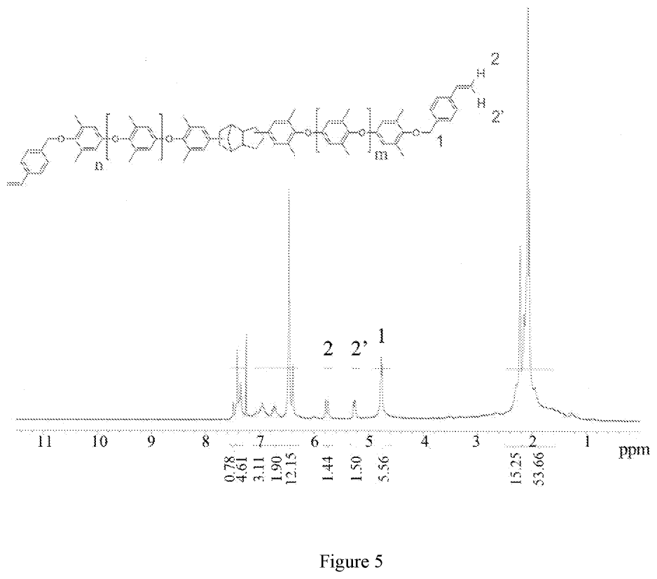

2.00 g of PPO bisphenol oligomer III prepared by embodiment 1, 0.1780 g of NaOH, 0.4948 g of p-vinylbenzyl chloride, and 20 mL of DMAc were added into a 150 mL three-necked flask. The mixture was stirred and heated to 90° C. under nitrogen atmosphere. After the reaction lasted for 1 hour, the reaction solution was instilled into 250 mL of methanol to precipitate and the precipitate solution was filtered to collect the powder. The filter cake was then washed, purified, and dried to give a light tan powder. According to the ¹H-NMR spectrum in FIG. 5, the characteristic peak at 4.2 ppm corresponding to both sides of the terminal phenolic groups of PPO bisphenol oligomer III was disappeared and the characteristic peaks at 5.2 ppm and 5.8 ppm corresponding to the unsaturated C═C double bonds of the oligomer III-vbe were observed. The number-average molecular weight was 4910 g/mol and the weight-average molecular weight was 7271 g/mol, which were analyzed by gel permeation chromatography. The solubility in organic solvents (50 wt %) and the molecular weights of the oligomer III-vbe provided by the present invention are summarized in Table 3 and Table 7.

TABLE 3

| | Embodi- | Embodi- | Embodi- | Embodi- | Com- |
|---|---|---|---|---|---|
| | ment | ment | ment | ment | parison |
| | 19 | 20 | 21 | 22 | 5 |
| Methanol | 48.6/1.5 | 48.6/5 | 48.6/10 | 48.6/0 | 48.6/30 |
| (mL)/water | | | | | |
| (mL) | | | | | |
| Mn/PDI | 4910/1.48 | 3187/1.57 | 2730/1.24 | 5250/2.80 | 1982/1.30 |
| Toluene | ++ | ++ | ++ | +− | ++ |
| Butanone | ++ | ++ | ++ | +− | ++ |
| Xylene | ++ | ++ | ++ | +− | ++ |

^{a}++ clear; +− slightly blurred

### Embodiment 20: Synthesis of Oligomer III-vbe

The oligomer III-vbe was prepared as embodiment 19 except that the PPO bisphenol oligomer III prepared by embodiment 1 was replaced with the PPO bisphenol oligomer III prepared by embodiment 4. The number-average molecular weight was 3187 g/mol and the weight-average molecular weight was 5013 g/mol, which were analyzed by gel permeation chromatography.

### Embodiment 21: Synthesis of Oligomer III-vbe

The oligomer III-vbe was prepared as embodiment 19 except that the PPO bisphenol oligomer III prepared by embodiment 1 was replaced with the PPO bisphenol oligomer III prepared by embodiment 5. The number-average molecular weight was 2730 g/mol and the weight-average molecular weight was 3376 g/mol, which were analyzed by gel permeation chromatography.

### Embodiment 22: Synthesis of Oligomer III-vbe

The oligomer III-vbe was prepared as embodiment 19 except that the PPO bisphenol oligomer III prepared by embodiment 1 was replaced with the PPO bisphenol oligomer III prepared by embodiment 6. The number-average molecular weight was 5250 g/mol and the weight-average molecular weight was 14700 g/mol, which were analyzed by gel permeation chromatography.

Comparison 5: Synthesis of Oligomer III-vbe

The oligomer III-vbe was prepared as embodiment 19 except that the PPO bisphenol oligomer III prepared by embodiment 1 was replaced with the PPO bisphenol oligomer III prepared by comparison 1. The number-average molecular weight was 1982 g/mol and the weight-average molecular weight was 2576 g/mol, which were analyzed by gel permeation chromatography.

### Embodiment 23: Synthesis of Oligomer IV-vbe

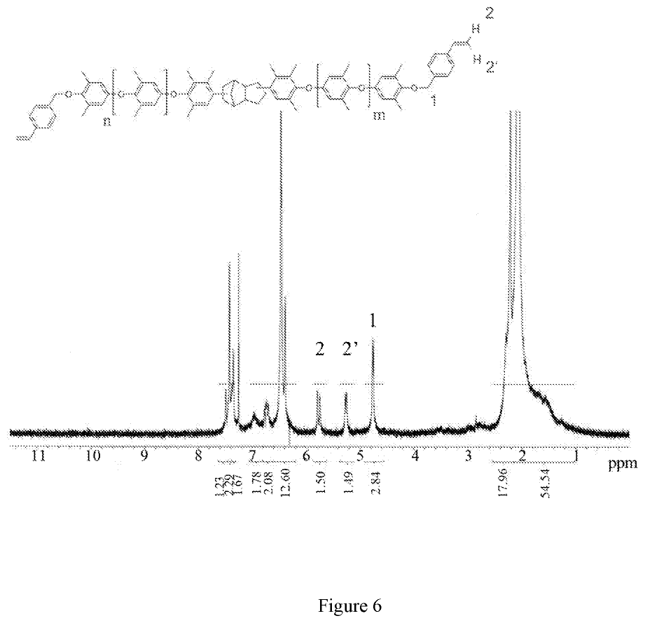

2.00 g of PPO bisphenol oligomer IV prepared by embodiment 7, 0.1780 g of NaOH, 0.4948 g of p-vinylbenzyl chloride, and 20 mL of DMAc were added into a 150 mL three-necked flask. The mixture was stirred and heated to 90° C. under nitrogen atmosphere. After the reaction lasted for 1 hour, the reaction solution was instilled into 250 mL of methanol to precipitate and the precipitate solution was filtered to collect the powder. The filter cake was then washed, purified, and dried to give a light tan powder. According to the ¹H-NMR spectrum in FIG. 6, the characteristic peak at 4.2 ppm corresponding to both sides of the terminal phenolic groups of PPO bisphenol oligomer IV was disappeared and the characteristic peaks at 5.2 ppm and 5.8 ppm corresponding to the unsaturated C═C double bonds of the oligomer IV-vbe were observed. The number-average molecular weight was 4128 g/mol and the weight-average molecular weight was 5741 g/mol, which were analyzed by gel permeation chromatography. The solubility in organic solvents (50 wt %) and the molecular weights of the oligomer IV-vbe provided by the present invention are summarized in Table 4 and Table 7.

TABLE 4

| | Embodi- | Embodi- | Embodi- | Embodi- | Com- |
|---|---|---|---|---|---|
| | ment | ment | ment | ment | parison |
| | 23 | 24 | 25 | 26 | 6 |
| Methanol | 48.6/1.5 | 48.6/5 | 48.6/10 | 48.6/0 | 48.6/30 |
| (mL)/water | | | | | |
| (mL) | | | | | |
| Mn/PDI | 4128/1.39 | 2909/1.35 | 2620/1.18 | 5520/2.70 | 1721/1.36 |
| Toluene | ++ | ++ | ++ | +− | ++ |
| Butanone | ++ | ++ | ++ | +− | ++ |
| Xylene | ++ | ++ | ++ | +− | ++ |

^{a}++ clear; +− slightly blurred

### Embodiment 24: Synthesis of Oligomer IV-vbe

The oligomer IV-vbe was prepared as embodiment 23 except that the PPO bisphenol oligomer IV prepared by embodiment 7 was replaced with the PPO bisphenol oligomer IV prepared by embodiment 8. The number-average molecular weight was 2909 g/mol and the weight-average molecular weight was 3930 g/mol, which were analyzed by gel permeation chromatography.

### Embodiment 25: Synthesis of Oligomer IV-vbe

The oligomer IV-vbe was prepared as embodiment 23 except that the PPO bisphenol oligomer IV prepared by embodiment 7 was replaced with the PPO bisphenol oligomer IV prepared by embodiment 9. The number-average molecular weight was 2620 g/mol and the weight-average molecular weight was 3090 g/mol, which were analyzed by gel permeation chromatography.

### Embodiment 26: Synthesis of Oligomer IV-vbe

The oligomer IV-vbe was prepared as embodiment 23 except that the PPO bisphenol oligomer IV prepared by embodiment 7 was replaced with the PPO bisphenol oligomer IV prepared by embodiment 10. The number-average molecular weight was 5520 g/mol and the weight-average molecular weight was 14904 g/mol, which were analyzed by gel permeation chromatography.

Comparison 6: Synthesis of Oligomer IV-vbe

The oligomer IV-vbe was prepared as embodiment 23 except that the PPO bisphenol oligomer IV prepared by embodiment 7 was replaced with the PPO bisphenol oligomer IV prepared by comparison 2. The number-average molecular weight was 1721 g/mol and the weight-average molecular weight was 2337 g/mol, which were analyzed by gel permeation chromatography.

### Embodiment 27: Synthesis of Cured Product of Oligomer III-mma (or IV-mma) with Epoxy Resin

The curing process of the oligomer III-mma (or IV-mma) prepared by embodiment 11 (or 15) with a commercial epoxy resin, HP 7200, was prepared as described below: the epoxy resin and the oligomer III-mma (or IV-mma) were added equivalence ratio of 1:1 into xylene as a solvent to form a solid content of 20 wt %. Additionally, DMAP as a hardener and tert-butyl cumyl peroxide (TBCP) as an initiator were added the epoxy resin of 2 wt % into the solution, respectively. The solution was subsequently poured into the mold with temperature programming as follows: at 80° C. for 12 hours and then at 180° C., 200° C., 220° C. for each 2 hours, and then cooled down to obtain a cured product of C-III-mma (or C-IV-mma) in brown color after mold release.

Comparison 7: Synthesis of Cured Product of SA9000 with Epoxy Resin

The curing process of SA9000 with a commercial epoxy resin, HP 7200, was as described below: the epoxy resin and SA9000 were added equivalence ratio of 1:1 into xylene as a solvent to form a solid content of 20 wt %. Additionally, DMAP and TBCP were added the epoxy resin of 2 wt % into the solution, respectively. The solution was subsequently poured into the mold with temperature programming as follows: at 80° C. for 12 hours and then at 180° C., 200° C., 220° C. for each 2 hours, and then cooled down to obtain a cured product of C-SA9000 in yellow color after mold release.

### Embodiment 28: Synthesis of Cured Product of Oligomer III-vbe (or IV-vbe)

The oligomer III-vbe (or IV-vbe) prepared by embodiment 19 (or 23) was added into xylene to form a solid content of 20 wt %. Additionally, TBCP was added the oligomer III-vbe (or IV-vbe) of 2 wt % into the solution. The solution was subsequently poured into the mold with temperature programming as follows: at 80° C. for 12 hours and then at 180° C., 200° C., 220° C. for each 2 hours, and then cooled down to obtain a cured product of C-III-vbe (or C-IV-vbe) in brown color after mold release.

Comparison 8: Synthesis of Cured Product of OPE-2st

OPE-2st was added into xylene to form a solid content of 20 wt %. Additionally, TBCP was added OPE-2st of 2 wt % into the solution. The solution was subsequently poured into the mold with temperature programming as follows: at 80° C. for 12 hours and then at 180° C., 200° C., 220° C. for each 2 hours, and then cooled down to obtain a cured product of C-OPE-2st after mold release.

Analysis Method

Thermogravimetric Analysis (TGA) was performed using Thermo Cahn VersaTherm under nitrogen and air with flow rate 20 mL/min.

Dynamic Mechanical Analysis (DMA) was performed using Perkin-Elmer Pyris Diamond. The cured products were cut to give specimens with 20 mm length, 10 mm width and 2 mm thickness. The storage modulus E′ and Tan δ were measured at a frequency of 1 Hz with heating rate of 5° C./min.

Thermal Mechanical Analysis (TMA) was performed using Perkin-Elmer Pyris Diamond with heating rate of 5° C./min.

400 MHz Nuclear Magnetic Resonance (NMR) Analysis was performed using Varian Unity Inova-600, DMSO-d6 at chemical shift of δ=2.49 ppm

Gel Permeation Chromatography (GPC) was performed using Hitachi L2400. 25 μL of sample solution was filtered with 0.22 μm filter and injected into the instrument to measure the number-average molecular weight, weight-average molecular weight (Mw) and polydispersity index (PDI) of samples.

Matrix-Assisted Laser Desorption/Ionization Time of Flight (MALDI-TOF MS) analysis was performed using Bruker Autoflex Speed. 5 mg of sample was dissolved in 1 mL toluene to give a sample solution. Then, 1 μL of sample solution mixed with 5 μL of matrix solution, and 5 μL of the mixed solution was deposit onto target plate. The molecular weight of sample was measured with 355 nm laser.

### Physical Analysis of the Cured Product, C-III-mma or C-IV-mma Prepared as in Embodiment 27

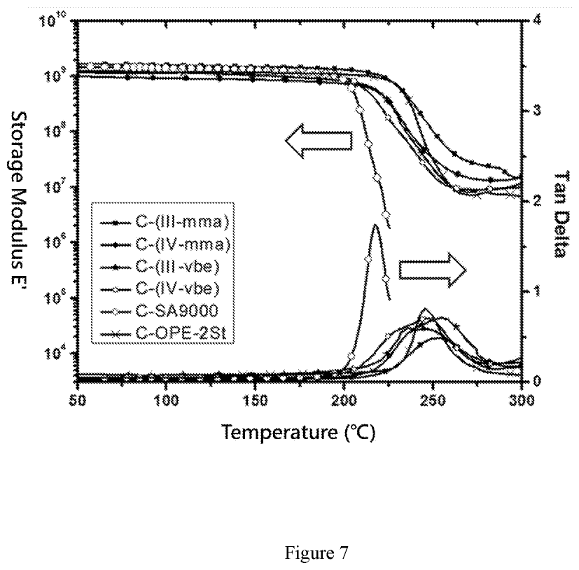

The glass transition temperatures of C-III-mma and C-IV-mma, measured by DMA, are 248° C. and 255° C., respectively. However, the glass transition temperature of C-SA9000 prepared by comparison 7 is 226° C., as shown in FIG. 7, which is pretty close to the solder temperature commonly used today. It may cause the substrate to bend after being heated and is not conducive to making double-sided PCB. However, the glass transition temperatures of C-III-mma and C-IV-mma prepared by the present invention are 248° C. and 255° C., respectively, which are at least 30° C. higher than the solder temperature commonly used today and can be sure that the substrate won't bend after being heated above the glass transition temperature. Another noteworthy is that the elastic modulus of C-III-mma and C-IV-mma are kept at 10⁷ GPa when being heated to 300° C., but the specimens of C-SA9000 are broken at 230° C. The results show that the cured products prepared by the present invention have better dimensional stability at high temperature. In FIG. 7, the upper half curves correspond to left y-axis and the lower half curves correspond to right y-axis.

Thermal stability of the cured products was analyzed by TGA. The decomposition temperatures at 5% weight loss (T_{d5%}) of C-III-mma and C-IV-mma are 405° C. and 393° C., respectively. The char yields at 800° C. of C-III-mma and C-IV-mma are 25% and 21%, respectively. Lastly, the electrical properties of C-III-mma and C-IV-mma were measured at a frequency of 1 Hz shown in Table 4. The Dk of C-III-mma and C-IV-mma are 2.86 and 3.3, respectively and the Df of C-III-mma and C-IV-mma are 3.3×10⁻³ and 3.8×10⁻³, respectively. Both Dk and Df values of C-III-mma and C-IV-mma are similar to those of C-SA9000.

In summary, the present invention provides the poly(2,6-dimethyl phenylene oxide) oligomers containing DCPD structure and followed by modifying the terminal groups of them to make the oligomers have active ester groups. As the functionalized poly(2,6-dimethyl phenylene oxide) oligomers are cured with epoxy resin, the secondary alcohols, generated after ring-opening of epoxy resin, are replaced by ester which is beneficial to lower the dielectric constant. Besides, the rigid aliphatic structure of DCPC and unsaturated C═C double bonds in the oligomer structure make hydrophobicity, lower electrical properties, and increase the rigidity of the cured products after the cross-linking reaction. Therefore, the cured products prepared by the functionalized poly(2,6-dimethyl phenylene oxide) oligomers in the present invention have high glass transition temperatures, high thermal stability and low dielectric properties.

### Physical Analysis of the Cured Product, C-III-vbe or C-IV-vbe Prepared as in Embodiment 28

The glass transition temperatures of C-III-vbe and C-IV-vbe, measured by DMA, are 253° C. and 244° C. respectively, and both of which are also at least 30° C. higher than the solder temperature commonly used today. Next, thermal stability of the cured products was analyzed by TGA. The decomposition temperatures at 5% weight loss (T_{d}5%) of C-III-vbe and C-IV-vbe are 426° C. and 415° C., respectively. The char yield at 800° C. of C-III-vbe and C-IV-vbe are 20% and 24%, respectively. Lastly, the electrical properties of C-III-vbe and C-IV-vbe were measured at a frequency of 1 Hz shown in Table 5. The Dk of C-III-vbe and C-IV-vbe are 2.60 and 2.48, respectively and the Df of C-III-vbe and C-IV-vbe are 3.0×10⁻³ and 3.2×10⁻³, respectively. The Dk and Df of C-OPE-2st prepared by comparison 8 are 2.64 and 7×10⁻³, respectively. It shows that the cured products prepared by the functionalized poly(2,6-dimethyl phenylene oxide) oligomers in the present invention have lower electrical properties. In summary, modifying the terminal groups of the poly(2,6-dimethyl phenylene oxide) oligomer by the styrene structure in the present invention makes the cured product have lower polarity. Thus, the cured products prepared by the oligomers III-vbe and IV-vbe have excellent thermal properties and dielectric properties, including the Dk can be reached to 2.48, and the glass transition temperature is or higher than 244° C. (even 253° C.), and the thermal decomposition temperature can be reached to 426° C.

TABLE 5

| | | Code name | Glass | | | |
|---|---|---|---|---|---|---|
| | Source of | of the | transition | | | |
| | raw | cured | temperature | | Dₖ | D_{f} |
| | materials | products | (° C.) | T_{d5%} (° C.) | (1 GHz) | (1 GHz) |
| Embodiment | Embodiment | C-III-mma | 248 | 405 | 2.86 | 0.0033 |
| 27 | 11 | | | | | |
| Embodiment | Embodiment | C-IV-mma | 255 | 393 | 2.88 | 0.0038 |
| 27 | 15 | | | | | |
| Comparison 7 | SA9000 | C-SA9000 | 226 | 412 | 2.85 | 0.0032 |
| Embodiment | Embodiment | C-III-vbe | 253 | 426 | 2.60 | 0.0030 |
| 28 | 19 | | | | | |
| Embodiment | Embodiment | C-IV-vbe | 244 | 415 | 2.48 | 0.0032 |
| 28 | 23 | | | | | |
| Comparison 8 | OPE-2st | C-OPE-2st | 222 | 365 | 2.64 | 0.0070 |

Moreover, PCB substrates (the formulation includes the PPO resin provided by the present disclosure, initiator, flame retardant, cross-linking agent and filler etc.) were also prepared by using the oligomer III-mma, oligomer IV-mma, oligomer III-vbe or oligomer IV-vbe in the present disclosure, respectively. According to the data shown in Table 6 and Table 7, the substrates made from the oligomer with the number-average molecular weight lower than 2500 g/mol couldn't pass the pressure cook test plus 288° C. solder dipping test (20 s dipping/20 s out, repeated three times for the same area of the substrate); only when the substrates made from the oligomer with the molecular weight higher than 2500 g/mol could pass the test. Besides, according to the data shown in Table 6 and Table 7, the molecular weight of the oligomer has also effects on glass transition temperature, thermal decomposition temperature and dielectric properties of the substrates. As the oligomers have higher molecular weight, the cured products have obviously the characteristics of poly(2,6-dimethyl phenylene oxide), and exhibit higher glass transition temperature, higher thermal stability, and also lower dielectric constant and dielectric loss shown in Table 6 and Table 7. As mentioned above, the molecular weight of the functionalized poly(2,6-dimethyl phenylene oxide) oligomer should be at least higher than 2500 g/mol in order to obtain a PCB substrate with excellent properties.

TABLE 6

| | Source of | | | | | |
|---|---|---|---|---|---|---|
| | bisphenol | molecular | | | | |
| | monomer and | weight of | | | | PCT 3 h + |
| | molecular weight, | functionalized | T_{g} | T_{d5} | Dₖ/D_{f} | 288° C. solder |
| Embodiment | Mₙ/PDI | mma, Mₙ/PDI | (° C.) | (° C.) | (10 GHz) | dipping |
| Embodiment | Embodiment 1, | 4045/1.39 | 239 | 399 | 3.10/0.003 | Pass |
| 11 | 3845/1.34 | | | | | |
| Embodiment | Embodiment 4, | 3130/1.31 | 230 | 397 | 3.20/0.004 | Pass |
| 12 | 2810/1.29 | | | | | |
| Embodiment | Embodiment 5, | 2711/1.25 | 225 | 385 | 3.22/0.003 | Pass |
| 13 | 2512/1.22 | | | | | |
| Embodiment | Embodiment 6, | 4563/2.60 | 247 | 410 | 3.12/0.004 | Pass |
| 14 | 4444/2.10 | | | | | |
| Comparison 3 | Comparison 1, | 1920/1.20 | 201 | 372 | 3.30/0.008 | Not pass |
| | 1719/1.20 | | | | | |
| Embodiment | Embodiment 7, | 3833/1.31 | 235 | 397 | 3.08/0.003 | Pass |
| 15 | 3113/1.17 | | | | | |
| Embodiment | Embodiment 8, | 2951/1.35 | 227 | 399 | 3.14/0.003 | Pass |
| 16 | 2670/1.20 | | | | | |
| Embodiment | Embodiment 9, | 2656/1.14 | 221 | 390 | 3.20/0.004 | Pass |
| 17 | 2347/1.10 | | | | | |
| Embodiment | Embodiment 10, | 5451/2.70 | 241 | 407 | 3.05/0.003 | Pass |
| 18 | 5312/2.50 | | | | | |
| Comparison 4 | Comparison 2, | 1712/1.36 | 198 | 370 | 3.42/0.007 | Not pass |
| | 1583/1.25 | | | | | |

TABLE 7

| | Source of bisphenol | Molecular | | | | PCT 3 h + |
|---|---|---|---|---|---|---|
| | monomer and | weight of | | | | 288° C. |
| Embodiment | molecular weight, | functionalized | T_{g} | T_{d5} | Dₖ/D_{f} | solder |
| for syntheses | Mₙ/PDI | vbe, Mₙ/PDI | (° C.) | (° C.) | (10 GHz) | dipping |
| Embodiment | Embodiment 1, | 4910/1.48 | 282 | 402 | 3.15/0.004 | Pass |
| 19 | 3845/1.34 | | | | | |
| Embodiment | Embodiment 4, | 3187/1.57 | 276 | 395 | 3.25/0.003 | Pass |
| 20 | 2810/1.29 | | | | | |
| Embodiment | Embodiment 5, | 2730/1.24 | 255 | 384 | 3.30/0.005 | Pass |
| 21 | 2512/1.22 | | | | | |
| Embodiment | Embodiment 6, | 5250/2.80 | 291 | 407 | 3.20/0.003 | Pass |
| 22 | 4444/2.10 | | | | | |
| Comparison 5 | Comparison 1, | 1982/1.30 | 231 | 377 | 3.32/0.009 | Not pass |
| | 1719/1.20 | | | | | |
| Embodiment | Embodiment 7, | 4128/1.39 | 280 | 404 | 3.22/0.003 | Pass |
| 23 | 3113/1.17 | | | | | |
| Embodiment | Embodiment 8, | 2909/1.35 | 279 | 395 | 3.15/0.004 | Pass |
| 24 | 2670/1.20 | | | | | |
| Embodiment | Embodiment 9, | 2620/1.18 | 249 | 382 | 3.16/0.005 | Pass |
| 25 | 2347/1.10 | | | | | |
| Embodiment | Embodiment 10, | 5520/2.70 | 289 | 410 | 3.18/0.003 | Pass |
| 26 | 5312/2.50 | | | | | |
| Comparison 6 | Comparison 2, | 1721/1.36 | 241 | 370 | 3.50/0.010 | Not pass |
| | 1583/1.25 | | | | | |

The present invention incorporates rigid DCPD structure into poly(2,6-dimethyl phenylene oxide) oligomer, which contributes to enhance rigidity and hydrophobicity of the material and introduces various unsaturated group into the terminal of the oligomers to make the cured products (with epoxy resin as well as its self-cured product) have higher glass transition temperatures and lower electric properties. The characteristics are complying with the current demands for making high frequency substrates. In addition to high frequency substrates, the applications of the functionalized poly(2,6-dimethyl phenylene oxide) oligomers provided by the present disclosure also include high-temperature additives, coating materials and adhesives etc.

It will be clear that various modifications and variations can be made to the disclosed methods and materials. It is intended that the specification and examples be considered as exemplary only, with the true scope of the disclosure being indicated by the following claims and their equivalents.

What is claimed is:

1. A functionalized poly(2,6-dimethyl phenylene oxide) oligomer containing dicyclopentadiene (DCPD), having a structure represented by Formula (I): 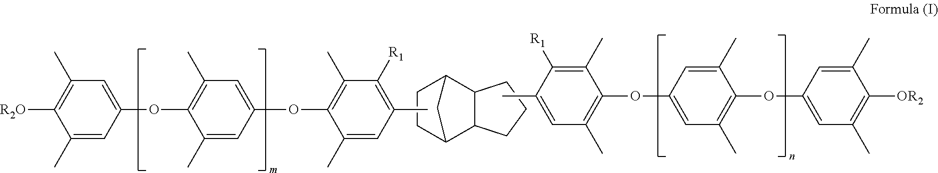
wherein n and m each independently represents a natural number; and the functionalized poly(2,6-dimethyl phenylene oxide) oligomer has a number-average molecular weight (Mn) from 2500 to 6000 g/mol;
each R₁ is independently H, C₁-C₆ alkyl group or phenyl group;
each R₂ is independently H, 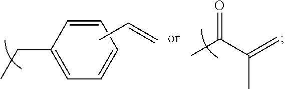
a cured product of the functionalized poly(2,6-dimethyl phenylene oxide) oligomer has a glass transition temperature equal to or higher than 244° C.

2. A method for producing the functionalized poly(2,6-dimethyl phenylene oxide) oligomer as claimed in claim 1 comprising:
(a) heating and agitating a reaction solution comprising DCPD, a phenol compound and a Lewis acid catalyst to 80-150° C.; after a synthesis is completed, washing, neutralizing and purifying the reaction solution, and then a bisphenol monomer is obtained;
(b) mixing the bisphenol monomer and a 2,6-dimethylphenol with a methanol/water co-solvent in the presence of a copper catalyst and a amine catalyst to carry out oxidative coupling polymerization under an oxygen atmosphere and a controlled temperature, and then a poly(2,6-dimethyl phenylene oxide) oligomer is obtained; wherein the controlled temperature is between 0 and 70° C.;
(c) reacting the poly(2,6-dimethyl phenylene oxide) oligomer with a methacrylic anhydride or a vinylbenzyl halide in the presence of an alkaline catalyst at 45-100° C., and then the functionalized poly(2,6-dimethyl phenylene oxide) oligomer is obtained; and
(d) copolymerizing the functionalized poly(2,6-dimethyl phenylene oxide) oligomer with a peroxide or an epoxy resin, and then a cured product of the functionalized poly(2,6-dimethyl phenylene oxide) oligomer is obtained.

3. The method as claimed in claim 2, wherein in the step (a), the mole ratio of the DCPD to the phenol compound is 1:2˜1:10; wherein in the step (b), the feed mole ratio of the bisphenol monomer to the 2,6-DMP is 1:2˜1:10.

4. The method as claimed in claim 2, wherein the phenol compound is phenol, 2,6-dimethylphenol or 2,3,6-trimethylphenol; the Lewis acid catalyst is BF₃ or an aluminum halide; the aluminum halide is aluminum trichloride, aluminum tribromide, aluminum ethyl dichloride or diethylaluminum chloride.

5. The method as claimed in claim 2, wherein as the phenol compound is 2,6-dimethylphenol, an unreacted 2,6-dimethylphenol is kept in the bisphenol monomer after the reaction solution is purified; and
in the step (b), directly mixing the bisphenol monomer and the methanol/water co-solvent without loading the 2,6-dimethylphenol in the presence of the copper catalyst and the amine catalyst to carry out oxidative coupling polymerization under the oxygen atmosphere and the controlled temperature.

6. The method as claimed in claim 2, wherein as the phenol compound is 2,6-dimethylphenol, an unreacted 2,6-dimethylphenol is kept in the bisphenol monomer after the reaction solution is purified; and
in the step (b), mixing the bisphenol monomer and the methanol/water co-solvent with an appropriate amount of the 2,6-dimethylphenol to carry out oxidative coupling polymerization in the presence of the copper catalyst and the amine catalyst.

7. The method as claimed in claim 2, wherein a water content of the methanol/water co-solvent is from 0% to 30%.

8. The method as claimed in claim 2, wherein a water content of the methanol/water co-solvent is from 0.5% to 20%.

9. The method as claimed in claim 2, wherein the copper catalyst is selected from the group consisting of CuCl, CuCl₂, CuBr, CuBr₂ and mixtures thereof; the amine catalyst is tertiary amine ((C₂H₅)₃N) or dialkylaminopyridine, and alkyl of the dialkylaminopyridine is C₁-C₆ alkyl group.

10. The method as claimed in claim 2, wherein a pressure of the oxygen atmosphere is from 14 psi to 150 psi, and a proportion of an oxygen content under the oxygen atmosphere is from 1% to 100%.

11. The method as claimed in claim 2, wherein the alkaline catalyst is selected from the group consisting of potassium carbonate (K₂CO₃), sodium carbonate (Na₂CO₃), potassium to hydroxide (KOH), sodium hydroxide (NaOH), sodium bicarbonate (NaHCO₃), sodium acetate, 4-dimethylamino pyridine, pyridine and mixtures thereof.

12. The method as claimed in claim 2, wherein the vinylbenzyl halide is selected from the group consisting of o-vinylbenzyl chloride, m-vinylbenzyl chloride, p-vinylbenzyl chloride, o-vinylbenzyl bromide, m-vinylbenzyl bromide, p-vinylbenzyl bromide and mixtures thereof.

13. A product comprising the functionalized poly(2,6-dimethyl phenylene oxide) oligomer as claimed in claim 1, wherein the product is selected from the group consisting of a high-frequency substrate, a high-temperature additive, a coating material, an adhesive and mixtures thereof.
